(19) United States
(12) Patent Application Publication
(10) Pub. No.: US 2022/0160583 A1
(43) Pub. Date: May 26, 2022

(54) FLUID TRANSFER SYSTEM AND COMPONENTS THEREFOR

(71) Applicant: TRENTA2 S.R.L., Bolzano (IT)

(72) Inventor: Dietrich Gallmetzer, Bolzano (IT)

(73) Assignee: TRENTA2 S.R.L., Bolzano (IT)

(21) Appl. No.: 17/617,072

(22) PCT Filed: Jun. 12, 2021
(86) PCT No.: PCT/EP2020/066315

§ 371 Date: Dec. 7, 2021

(30) Foreign Application Priority Data
Jun. 13, 2019 (EP) 19180089.5

(51) Int. Cl. A61J 1/20; A61J 1/14; A61J 1/10

(52) U.S. Cl. CPC A61J 1/2096; A61J 1/2013; A61J 1/10; A61J 1/2048; A61J 1/1418; A61J 1/2072

(57) ABSTRACT

The invention relates to a fluid transfer system (1) for transferring medical substances or drugs. It further relates to components and parts of such a transfer system (1), in particular its connecting components (2, 6) and the parts equipped with such connecting components (2, 6), such as syringes (4), containers (vials) (8) or application systems. According to the invention, a screw connection of the connecting components (2, 6) to each other is provided with regard to a particularly good dosability of the applied holding forces. In addition, a needle cap (2) made of comparatively soft, elastic material is provided as a seal and sealing closure for the hollow needles (40, 42) provided as actual transfer channels in the connection component (2).

### FIELD OF INVENTION

The invention relates to a liquid transfer system for the transfer of medical substances or drugs. It further relates to components and parts of such a transfer system, in particular its connection components and the parts equipped with such connection components, such as syringes, containers (vials) or application systems.

### BACKGROUND OF INVENTION

In modern medical procedures or therapies, drugs or substances may be used that are actually toxic or otherwise harmful or hazardous. For personnel handling such substances, such as pharmacists and nurses, this can pose acute and long-term health risks, especially when repeatedly exposed to drugs or solvents that may become airborne during preparation, drug administration and other similar operations. This problem can be particularly severe when cytotoxins, antiviral drugs, antibiotics or radiopharmaceuticals are involved. Potential health risks from exposure to these drugs include increased risk of cancer, genetic alterations, and the like. Furthermore, sampling or blood collection for viral infections or the like may also pose an increased risk to handling personnel.

When performing infusions, it is often necessary to inject a drug or active ingredient into the infusion fluid, into an infusion bag or into another infusion container. This is often done by piercing a septum or other fluid barrier of an injection port on the infusion bag or line with the needle of a syringe filled with the medical fluid in question. However, even prior to thes step, it may be necessary to transfer the medical fluid from a vial or vial into a syringe and therefrom the syringe into a secondary container. In each of these steps, personnel may come into contact with the medical fluid through contamination. This contamination can be vaporized medical fluid or aerosol in the air. The contaminants can contaminate the personnel through the lungs or through vaporized medical fluid or aerosol in the air that condenses on the skin and then penetrates the skin of the affected person. Some medications have even been known to penetrate protective gloves, thereby contaminating personnel.

On the one hand, for the above-mentioned reasons of health protection, and on the other hand, in view of the fact that drugs with an extremely high dose price have recently been approved, it is urgently desirable or even necessary to reliably prevent the release of even the smallest quantities of such drugs or active substances into the environment. For this purpose, so-called closed transfer systems are in use, in which, in the manner of an encapsulated design, it is ensured in each phase of the liquid transfer that the active substance or the gases or aerosols released by it cannot escape into the environment.

Such fluid transfer systems typically include a first connector component or adapter for connection to a first fluid container, such as a syringe, and a second connector component or adapter for connection to a drug container, vial, second syringe, or intravenous line that provides fluid access to the patient's circuit. For example, the clinician or treating person can connect a syringe to a vial via the syringe adapter and the associated vial adapter and aspirate the drug therein into the syringe. The adapters are then disconnected, and the syringe is connected to an intravenous line or access via the syringe adapter and an associated delivery adapter so that the drug in the syringe can be administered to the patient.

Such a fluid transfer system, including the necessary components such as connection adapters and the like, is known, for example, from WO2016199133A1.

### SUMMARY OF INVENTION

The invention is based on the task of providing a liquid transfer system for the transfer of medical substances or medicaments of the above-mentioned type, including its essential components such as, in particular, the connection components, with which the unintentional delivery or release of active ingredient components or gases or aerosols thereof is particularly reliably prevented while keeping handling particularly simple.

With regard to a first connection component, in particular for a medical syringe, this task is solved in accordance with the invention by means of a needle system comprising a number of hollow needles and arranged in a tubular connection socket surrounded by a flanged casing provided with a thread, and by means of an elastic needle cap arranged inside the connection socket and sealingly enclosing the distal tip(s) of the or each needle of the needle system in the unloaded state.

Advantageous embodiments of the invention as well as further aspects or variants considered to be independently inventive are the subject of the sub-claims and/or the following description of the figures.

The invention is based on the consideration that a stable and sealing connection between the respective connection components is particularly important for a safe and loss-free transfer of liquid between the syringe and a further container, for example a vial. For this purpose, on the one hand, a suitable seal must be provided at the interfaces or contact surfaces of both connection components, and on the other hand, a suitable frictional connection should be established between the connection components, on the one hand for the purpose of mechanically stabilizing the components relative to each other and, on the other hand, for the purpose of improving the sealing effect by pressing on. In the known systems, for example the one known from WO2016199133A1, a hooking or latching of the connecting components to each other is provided for this purpose, with suitably positioned swivel arms effecting the latching of the components.

Deviating from this design principle and with regard to a particularly good dosability of the applied holding forces, a screw connection of the connection components to each other is now provided, whereby the holding force between the connection elements can be modified and adjusted as required by suitable actuation of the screw connection. In addition, a needle cap made of comparatively soft, elastic material is provided as a seal and sealing closure for the hollow needles provided as actual transfer channels in the connection component. This is deformed and pushed back on contact with the other connecting element, so that the hollow needles inside pierce it and are thus exposed to the outside to establish the media-side connection. Depending on the setting of the screw connection, the deformation of the needle cap can vary, so that the sealing effect provided by the needle cap and its contact with the mating surface can be suitably optimized accordingly. After completion of the liquid transfer and after separation of the components, however, the needle cap resumes its original shape due to the elasticity of its base material and thus encloses the needle tips again, so that reliable enclosure and sealing of the syringe interior, including the needle outlets, is ensured even after completion of the transfer.

Very preferably, the needle cap is made of material with very good resistance to pressure deformation and particularly good pressure relaxation properties. A thermoplastic elastomer (TPE) is particularly preferred for this purpose.

In a very particularly advantageous embodiment, which is considered to be independently inventive, a spring element, especially preferably a spiral spring, is arranged inside the needle cap, which supports the restoration of the original shape of the needle cap after completion of the transfer. For example, a spiral spring surrounding the needle system can be arranged inside the needle cap, which is supported on the one hand by a base plate in which the needles of the needle system are held, and on the other hand by the cover surface of the needle cap. When the needle cap is deformed as a result of contact with the other connecting element, the spring element is deformed together with the needle cap and is pretensioned in the process. After completion of the fluid transfer and after separation of the components, the spring element relaxes again and thus supports the desired deformation of the needle cap until it resumes its original shape completely enclosing the exposed tips of the needle system.

In order to enable a completely encapsulated liquid transfer in which neither liquid losses nor an escape of gases or aerosols from the liquid to the environment have to be accepted, the needle system is advantageously designed as a double needle system. Analogous to the design known from WO2016199133A1, this comprises a first hollow needle intended for the actual liquid transfer and a second hollow needle intended for a gas transfer.

In order to enable a particularly reliable and safe connection on the media side by means of the needle system, in a further advantageous embodiment the flange jacket is designed to rotate freely around the needle system and around the needle cap enclosing it. This makes it possible to use the connection thread, with which the mechanically comparatively stable and thus particularly favorable screw connection of the connection components to one another is to be effected, in a particularly simple and favorable manner and, in particular, without disturbing or impairing the media-side connection via the needle system. This can be used in a particularly advantageous manner in order to be able to design the needles of the needle system with particularly fine dimensions, in particular comparatively thin-walled and with a comparatively small outer diameter.

With regard to a second connecting component, in particular for a container for medical substances or medicaments or for a transfer system for medical substances or medicaments, and in particular for connection to the first connecting component described above, the above-mentioned task is solved in an embodiment considered to be independently inventive by means of a connecting socket provided with a thread, in which a sealing plug consisting of a polypropylene, preferably of PP Purell HP570, is arranged.

In contrast to conventional systems, in which, for example, containers for medicinal substances or drugs are closed with a seal-like closure cap made of aluminum or the like, the container can be designed to be reclosable by means of such a second connecting component. In conventional systems, the seal-like closure lid is pierced by the needle system during initial use, so that it remains open after liquid transfer and separation of the components, so that the substance in the container must be completely transferred; any residues or even gases or aerosols remaining in the container must then be disposed of separately and in a safe and encapsulated system. In the present system, which is considered to be inventive, on the other hand, the design of the sealing plug results in the material resuming its original position after removal of the needle system, thus sealing the container again. Thus, to a certain extent, multiple use of the container and thus hatchwise removal of the active ingredient contained therein is possible.

To produce the intended screw connection of the components to one another, the thread of the second connecting component is advantageously adapted to the thread of the first connecting component in terms of design, dimensioning and the like. Very advantageously, the said components form a Luer thread.

A container for medical substances or medicaments equipped with such a second connecting component is also considered to be independently inventive. In a particularly advantageous embodiment, this is provided with an outer cap surrounding the connection component, which is provided with a disposable closure. This disposable closure, which may comprise, for example, a sealable cap that can be torn off or sealed, permits easy and reliable identification of whether or not the container has already been used for liquid transfer, and thus facilitates the assignment of whether or not the container has already been “opened” and thus should preferably be used for further liquid withdrawal until it is completely emptied and thus should be disposed of.

The invention further relates, in an embodiment considered to be independently inventive, to a medical syringe which is provided at its distal end with a connector component according to one of the embodiments described above. Furthermore, the invention relates in an embodiment also considered to be independently inventive to a fluid transfer system for the transfer of medical substances or medicaments with a first connection component of the embodiment described above and with a second connection component of the embodiment described above adapted thereto with regard to the dimensioning and design of the connection pieces.

### DESCRIPTION OF DRAWINGS

An embodiment of the invention is explained in more detail with reference to a drawing. Therein show:

FIG. 1 a longitudinal section of a medical syringe that can be connected to a container for drugs or medications via connection components of a fluid transfer system,

FIG. 2 the components as shown in FIG. 1 in longitudinal section in the connected state,

FIG. 3 an enlarged section of the components according to FIG. 2,

FIG. 4 a particularly preferred embodiment of a container for use in the liquid transfer system according to FIG. 1,

FIG. 5 in longitudinal, section, a particularly preferred embodiment of a first connection component for use in the fluid transfer system according to FIG. 1, in the state before the components are connected to each other,

FIG. 6 the connecting component according to FIG. 5 in longitudinal section with components connected to each other,

FIG. 7 a syringe adapter before assembly with an assigned syringe in longitudinal section,

FIG. 8 the syringe according to FIG. 7 with mounted syringe adapter,

FIG. 9 an infusion bag with adapter,

FIG. 10 the infusion bag provided with adapter according to FIG. 9 before assembly with an associated syringe in longitudinal section, and

FIG. 11 the syringe according to FIG. 10 with mounted infusion bag.

### DETAILED DESCRIPTION

Identical parts are provided with the same reference signs in all figures. The invention will be explained below primarily with reference to the embodiment example in which a medical syringe is equipped with a first connection component of the type mentioned and a container for medical substances or medicaments is equipped with a second connection com ponent of the type described. However, other embodiments are of course also conceivable and covered by the present invention, in which other components of a medical system, for example an access interface to an intravenous application system or an adapter for an infusion bag (“IV bag”), with such a second connection component, or also an “inverse arrangement”, in which a medical syringe is provided with the second connection component and the respective other component, for example a container or an access interface, is provided with such a first connection component. Equally conceivable and encompassed by the present invention are, of course, other embodiments in the non-medical field in which liquid transfers must be carried out under reliable gas or material exclusion without the handling persons being exposed to an explosion, for example in applications in process engineering or for hazardous chemical processes and the like.

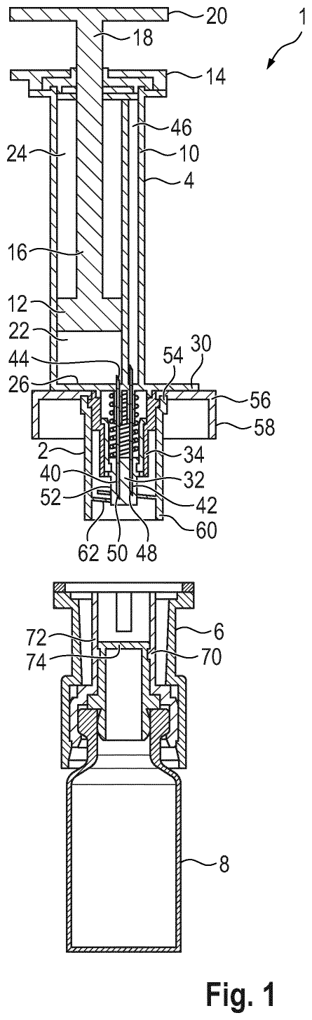

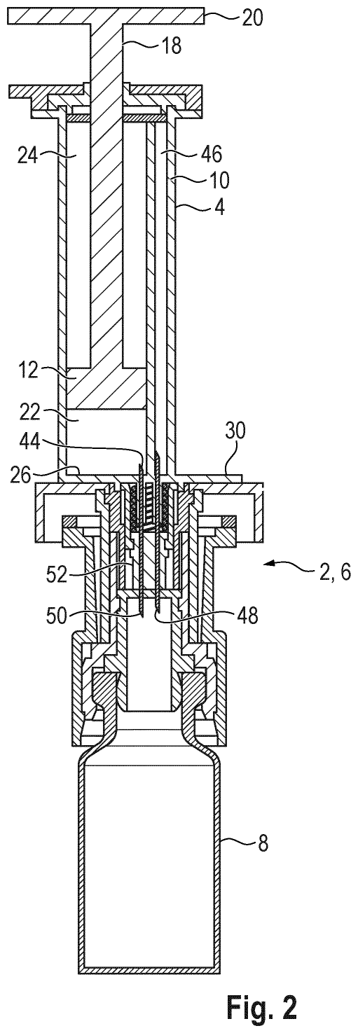

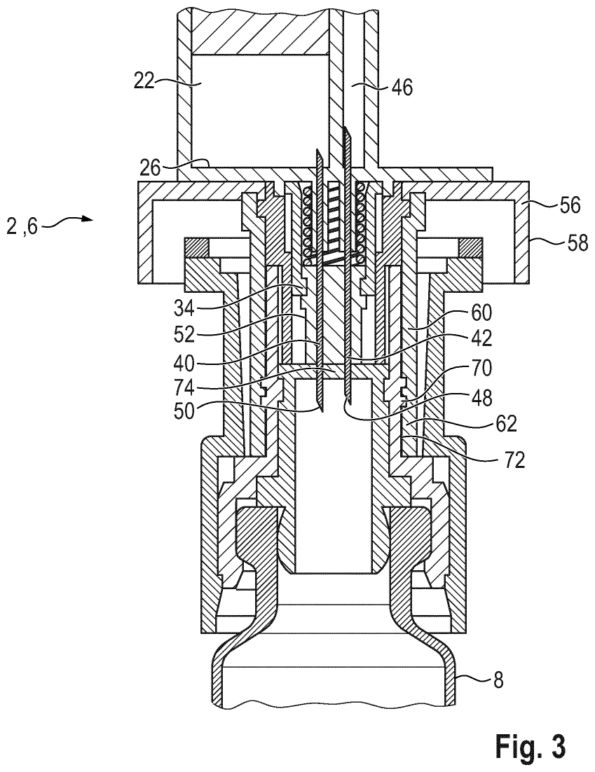

The fluid transfer system 1 according to the invention will thus be explained in the following on the basis of a medical syringe 4 provided with a first connection component 2 and a container 8 for medical substances or medicaments provided with a second connection component 6. In FIG. 1, the components, i.e., the syringe 4 and the container 8, are shown in longitudinal section in a separate, still separated state; FIGS. 2 and 3 show the components analogously (FIG. 3 in sections) in longitudinal section after the combination has been produced.

The syringe 4 comprises a cylindrical or tubular hollow body 10 forming a syringe housing, in which a plunger 12 whose outer dimensions are precisely adapted to the inner contour of the hollow body 10 is displaceably guided. The piston 12 is arranged at the end on an actuating plunger 16 guided in a sealing manner through a cover plate 14, which in turn is provided with a pusher plate 20 at its free end 18. The piston 12 divides the internal volume of the hollow body 10 into a distal partial volume 22 intended for receiving liquid and a proximal partial volume 24 intended for receiving gases or aerosols, the volume contents of which can be varied by positioning the piston 12 within the hollow body 10. The first connection component 2 is arranged at the distal end 26 of the hollow body 10 and thus of the distal partial volume 22.

The first connection component 2, which is regarded as independently inventive and which in the embodiment example is designed integrally with the hollow body 10 but could also be designed as a separate, retrofittable component, comprises a distal end plate 30 which sealingly closes off the distal partial volume 22 of the hollow body. The end plate 30 forms a needle holder for a needle system 32 and, on its side facing away from the partial volume 22, a tubular connecting piece 34 surrounding the needle system 32 for establishing a connection with the second connection component 6.

In the embodiment example, the needle system 32 is designed as a double needle system and comprises a first hollow needle 40 provided for liquid transfer and a second hollow needle 42 provided for gas transfer. The first hollow needle 40 projects with its inner end 44 into the partial volume 22 and thus forms a liquid channel for liquid exchange. The second hollow needle 42, on the other hand, is connected on the inside to a gas channel 46, which is guided in the manner of a bypass along the inner wall of the hollow body 10 into the proximal partial volume 24. Via the second hollow needle 42, gas or aerosol can thus be transferred from the distal needle tip 48 thereof into the proximal partial volume 24 in the manner of an encapsulated embodiment or can be guided from the latter towards the needle tip 48. Analogous to the mode of operation described in WO2016199133A1, liquid can thus be transferred from the container 4 into the distal partial volume 22 when the container 4 is connected and, at the same time, gas can be transferred from the proximal partial volume 24 into the container 4 without substances being discharged to the outside and with pressure compensation taking place automatically.

The first and second hollow needles 40, 42 protrude with their distal needle tips 48, 50 from the open end of the tubular connection nozzle 34. In order to ensure reliable encapsulation of the system, an elastic, comparatively easily deformable needle cap 52 is also fastened inside the connection socket 34, which sealingly encloses the distal tips 48, 50 of the hollow needles 40, 42 of the needle system in the unloaded state. In the particularly preferred embodiment shown in the embodiment example, the needle cap 52 consists of a thermoplastic elastomer (TPE). When a connection is made with the second connection component 6, in particular by pressing it against a contact plate provided, the needle cap 52 deforms, with the distal tips 48, 50 piercing the needle cap 52 and thus being exposed, so that an exchange of media is made possible.

On the outside, the connecting nozzle 34 is surrounded by a connecting sleeve 56 mounted on a circumferential rib 54. This is rotatably mounted about the needle system 32 and has an actuating rim 58, via which the user can effect a rotation, and a flange jacket 60, which is provided on the inside with a thread 62. In the embodiment, the thread 62 in combination with the flange jacket 60 is designed as a component of a Luer connection. In the embodiment example, the thread 62 is designed with only one thread or thread turn but can of course also have further threads and/or thread turns.

The container 8 for medical substances or medicines, also referred to as a vial, also shown in longitudinal section in FIG. 1, is correspondingly provided with a second connecting component 6 designed for connection to the first connecting component 2. This comprises a connecting socket 72 provided with a thread 70, in which a closure plug 74 made of the particularly preferred material PP Purell HP 570, which is considered to be independently inventive with regard to the choice of material, is arranged. The connecting piece 72 provided with the thread 70 is designed to match the flange jacket 60 and its thread 62, so that these components form a Luer connection with each other.

To produce the connection of the connection components 2, 6 to one another on the media side, as shown in the overall view in FIG. 2 and in enlarged sections in FIG. 3, the syringe 4 provided with the first connection component 2 is first brought into contact with the container 8 provided with the second connection component 6 in such a way that the thread 62 of the flange jacket 60 engages with the thread 70 of the connection socket 72. This creates a threaded connection between these components, which can be further tightened by rotation of the flange shell 60, i.e. actuation of the actuation rim 58. This further moves the first connector element 2, as seen in the longitudinal direction of the syringe 4, towards the second connector component 6 and thus the container 8. As soon as the end surface of the needle cap 52 comes into contact with the connecting plug 74 serving as a contact plate, the needle cap 52 begins to deform due to its elastic design, and the distal needle tips 48, 50 begin to penetrate the needle cap 52. As the threaded connection is further tightened, the distal needle tips 48, 50 completely penetrate the needle cap 52 and begin to penetrate the sealing plug 74.

When the screw connection is further actuated to the end position shown in FIGS. 2 and 3, the distal needle tips 48, 50 also completely penetrate the sealing plug 74. In this state, the first hollow needle 40 forms an open fluid channel and enables fluid exchange between the interior of the container 8 and the distal partial volume 22, and gas exchange between the proximal partial volume 24 and the interior of the container 8 is enabled via the second hollow needle 42 and the gas channel 46 connected to it. For lossless and encapsulated fluid transfer from the container 8 into the distal partial volume 22 of the syringe 4, starting from an initial state in which the piston 12 is positioned immediately adjacent to the distal end 26 of the hollow body 10, the piston 12 can now be moved within the hollow body 10 away from its distal end 26. This causes fluid to flow from the interior of the container 8 into the distal partial volume 22 of the syringe 4. At the same time, the gas in the proximal partial volume 24 is compressed so that it flows into the interior of the container 8 via the gas channel 46 and the second hollow needle 42 to equalize the pressure. This enables a safe and encapsulated exchange of media between syringe 4 and container 8 without liquid or gas escaping into the environment.

After removing the intended quantity of liquid from the container 8, the components are separated again by loosening the screw connection. The hollow needles 40, 42 of the needle system 32 are pulled out of the interior of the container 8 as seen in the longitudinal direction of the syringe 4. As soon as the needle tips 48, 50 have been completely withdrawn from the sealing plug 74, the latter resumes its original shape due to the choice of material and seals the interior of the container 8 from the outside again. As soon as the end surface of the needle cap 52 is also lifted off the sealing plug 74, which serves as a contact plate, the needle cap 52 begins to resume its original shape due to its material properties, so that the needle tips 48, 50 are again completely positioned in their interior space and thus the needle cap 52 again completely encloses the needle tips 48, 50. Thus, after the components have been separated from one another, complete enclosure of the interior of the container 8 on one side and of the interior of the hollow body 10 is again provided.

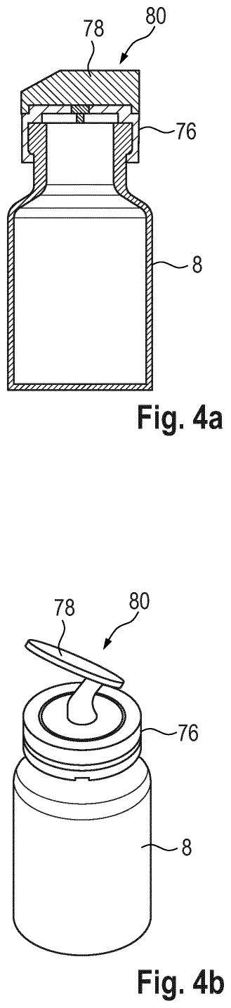

A particularly preferred design of the vial or container 8, which is considered to be independently inventive, is shown in longitudinal section in FIG. 4. As can be seen from the lateral view in FIG. 4a and also from the representation of the assembled article in perpective view in FIG. 4b, the container in this embodiment comprises an outer closure cap 76 surrounding the connection component 6 and provided with a one-way closure 78. In the embodiment example, the one-way closure 78 comprises a seal lid 80 that can be torn off, or alternatively or additionally, for example, a sealed seal lid 80 that is removed when the container 8 is opened for the first time. This design permits problem-free and reliable identification of whether or not the container 8 has already been used for liquid transfer, and thus facilitates the assignment of whether the container 8 has already been “opened” and should thus preferably be used for further liquid withdrawal until it is completely emptied and should thus be disposed of.

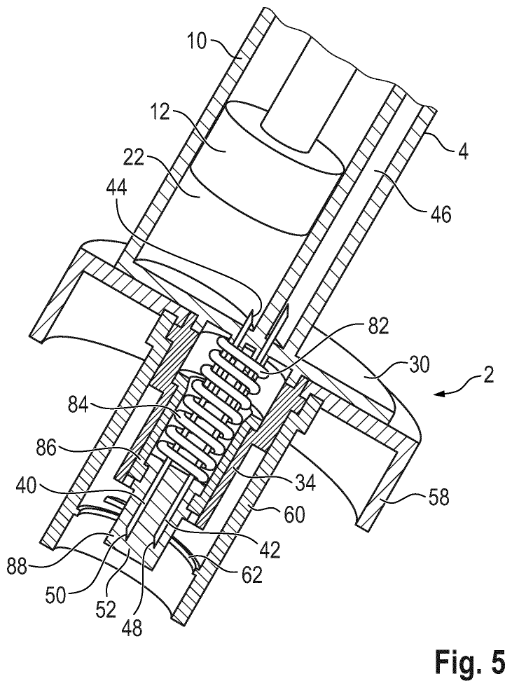

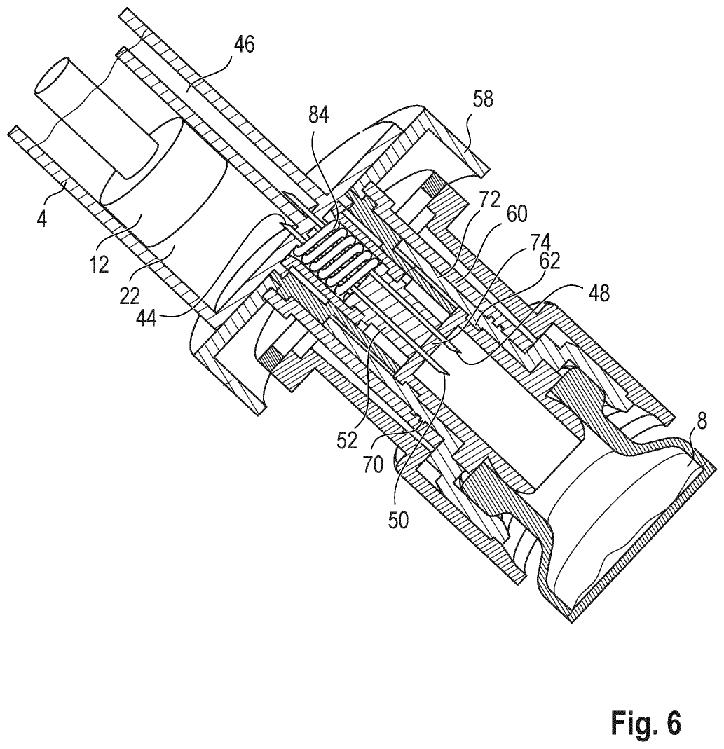

A very particularly advantageous design of the first connecting component 2, which is considered to be independently inventive, is shown in enlarged longitudinal section in FIG. 5 (state before the components are connected to one another) and in FIG. 6 (state with components connected to one another). As can be seen from this illustration, in this embodiment a spring element 82, in this example a spiral spring 84, is arranged as a further component within the needle cap 52. In this embodiment, the spiral spring 84 is arranged inside the needle cap 52, surrounding the needle system 32. On the one hand, it is supported on the cover plate 30 of the distal partial volume 22, in which the needles 40, 42 of the needle system 32 are held, and on the other hand, on the cover surface of the needle cap 52. In the embodiment shown, the needle cap 52 is also made in two parts and comprises, in addition to the actual cap body 86, a needle guard 88 attached distally thereto, whereby in this embodiment the spiral spring 84 is supported on the base of the needle guard 88.

In the state shown in FIG. 5 before the components are connected to each other, the spiral spring 84 is relaxed. However, as a result of the deformation of the needle cap 52 due to contact with the other connecting element 6, the spring element 82 is deformed together with the needle cap 52 and thereby pretensioned; this state is shown in FIG. 5. After completion of the fluid transfer and after separation of the components, the spring element 82 relaxes again and thus supports the desired deformation of the needle cap 52 until it resumes its original shape completely enclosing the exposed tips 48,50 of the needle system 32.

The medical syringe 4 described above, which is equipped with the first connection component 2, can be used, for example, for transferring fluids between different containers of the container 8 type or also for transferring fluids from such a container 8 to an intravenous (IV) access. In such applications, the medical syringe 4 is respectively connected via its connection component 2 to the associated second connection component 6 of the respective container 8 or to an access interface of the intravenous access, so that liquid can be drawn from the respective container 8 into the distal partial volume 22 of the syringe 4 or dispensed therefrom into a corresponding container 8 or the IV access.

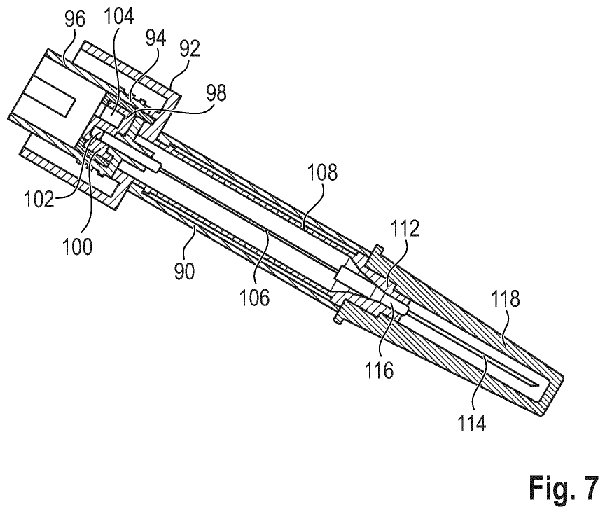

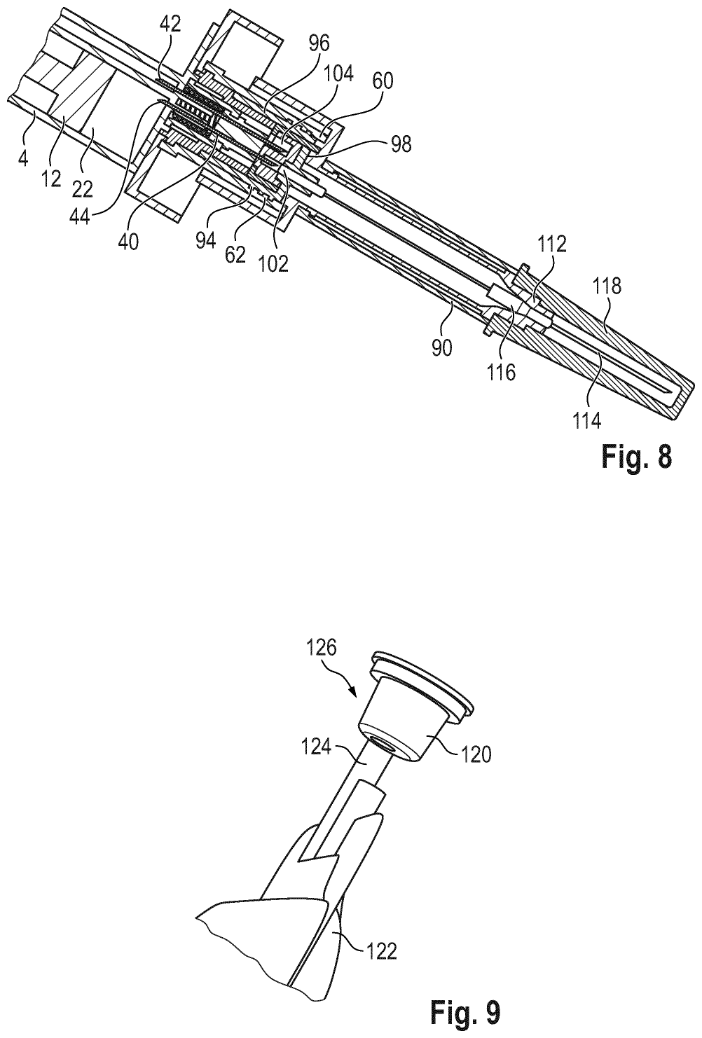

Alternatively, however, and in an embodiment considered to be independently inventive, after filling with the liquid transferred as a medicament or active ingredient, the syringe 4 can also be prepared for direct injection of the liquid and thus as an injection syringe. For this purpose, a syringe adapter 90 considered to be independently inventive is provided, as shown—in each case in longitudinal section together with the end region of the syringe 4—in FIG. 7 before and in FIG. 8 after assembly with the syringe 4.

As shown, the syringe adapter 90 shown in longitudinal section in FIGS. 7, 8 is designed for assembly to the medical syringe 4 using the first connection component 2 thereof. For this purpose, the syringe adapter 90 is correspondingly provided with a further connection component 92 suitably designed for connection to the first connection component 2. Analogous to the variants described above, this comprises a connection piece 96 provided with a thread 94, which—like the corresponding components of the second connection component 6—is designed to match the flanged sheath 60 of the first connection component 2 and its thread 62, so that these components form a Luer connection with one another.

A contact element 98 is arranged within the connection socket 96 for establishing a media-side connection with the needle system 32. The contact element 98 is substantially formed by a plug-like base body 100 having a cavity or hollow space 102 for receiving the needle tip 50 of the first hollow needle 40 provided for fluid transfer, and another cavity or hollow space 104 for receiving the needle tip 48 of the second hollow needle 42 connected on the media side to the gas channel 46. Encapsulated gas exchange is not provided in this case, and accordingly the further cavity 104 is of the blind hole type and is not connected to any further media channels.

In contrast, the cavity 102 is designed and provided as a transfer space for transferring the fluid fed from the distal partial volume 22 of the syringe 4 via the hollow needle 40. For this purpose, the proximal end of a transfer needle 106 opens into the cavity 102, which is arranged within a hollow body 108 of cylindrical or tubular design forming the housing of the syringe adapter 90. A needle holder 112 is attached to the front or distal end 110 of the hollow body 108, in which the hollow needle 114 intended for injection of the active agent is mounted in a hearing sleeve 116. The needle holder 112 could be made in one piece with the hollow body 108 forming the housing. In the embodiment example, however, the needle holder 112 is designed as a separate component in an embodiment that is considered to be independently inventive. The needle holder 112 is attached or attachable to the hollow body 108 forming the housing, but could also be designed to be screwed on by means of a thread, for example a Luer thread. The hollow body 108 is surrounded on the outside by a needle protection sleeve 118 which can be displaced in the longitudinal direction.

In a further alternative embodiment, which is also considered to be independently inventive, the syringe 4 can also be used for injecting the liquid into an infusion bag (“IV bag”) after filling with the liquid transferred as a drug or active ingredient. This allows the active ingredient or drug to be added to the actual infusion fluid, for example, or to be provided directly as an infusion fluid. From the infusion bag, the active ingredient can then be applied via an intravenous application system.

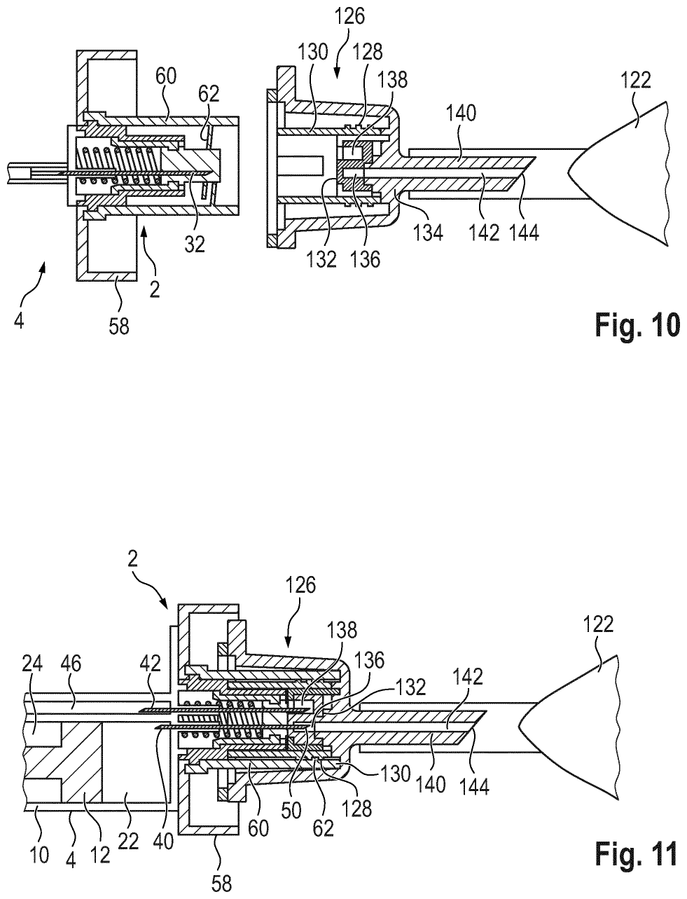

To make this possible, an adapter 120, which is considered to be independently inventive, is provided for an infusion bag 122, as shown together with the infusion bag 122 in perspective view in FIG. 9 and—in each case in longitudinal section together with the end region of the syringe 4—in FIG. 10 before and in FIG. 11 after assembly with the syringe 4.

The infusion bag 122 according to FIG. 9, also referred to as “IV bag”, is provided for connection to an intravenous application system, with which a liquid, for example a drug or also an isotonic liquid or the like, held in the infusion bag 122 can be administered to a patient as an infusion. The infusion bag 122 is provided with a connector 124 via which an exchange of media, in particular the introduction or removal of fluid into/from the interior of the infusion bag 122 is possible. In order to enable the use of the syringe 4 for liquid transfer into (or, if required, out of) the infusion bag 122, the adapter 120 is provided, which is shown in FIGS. 9 to 11 in a state already mounted on the connector 124 of the infusion bag.

The adapter 120 shown in longitudinal section in FIGS. 10, 11 is designed as shown for mounting on the medical syringe 4 using its first connector component 2. For this purpose, the adapter 120 is accordingly provided with a further connection component 126 suitably designed, for connection to the first connection component 2. Analogous to the variants described above, this comprises a connecting socket 130 provided, with a thread 128, which—like the corresponding components of the second connecting component 6 and the corresponding components of the further connecting component 92—is designed to be adapted to the flange jacket 60 of the first connecting component 2 and its thread 62, so that these components form a Luer connection with one another.

A contact element 132 is arranged within the connection socket 130 for establishing a media-side connection with the needle system 32. In a similar embodiment to the aforementioned contact element 98, the contact element 132 is substantially formed by a base body 134 having a cavity or hollow 136 for receiving the needle tip 50 of the first hollow needle 40 provided for fluid transfer and another cavity or hollow 138 for receiving the needle tip 48 of the second hollow needle 42 connected to the gas channel 46 on the media side. No encapsulated gas exchange is provided in this case, and accordingly the further cavity 138 is in the nature of a blind hole and is not connected to any further media channels.

In contrast, the cavity 136 is designed and provided as a transfer space for transferring the fluid fed from the distal partial volume 22 of the syringe 4 via the hollow needle 40. For this purpose, the cavity 136 goes into a media channel 142 arranged in a connection piece 140 or a grommet in the manner of an axial central bore, the free end 144 of which communicates on the media side with the interior of the infusion bag 122.

### REFERENCE LIST

1 Fluid transfer system

2 first connection component

4 Medical syringe

6 second connection component

8 Container for medical substances or medicines

10 Hollow body

12 Piston

14 Cover plate

16 Actuating plunger

18 free end

20 Handle plate

22,24 partial volume

26 distal end

30 end plate

32 needle system

34 connecting piece

40,42 hollow needle

44 inner end

46 Gas channel

48,50 needle tip

52 needle cap

54 rib

56 connecting sleeve

58 Actuating rim

60 Flange jacket

62 Thread

70 Thread

72 Connecting sleeve

74 Sealing plug

76 Closing cap

78 One-way cap

80 Sealing cap

82 Spring element

84 Coil spring

86 Cap body

88 Needle protection body

90 Syringe adapter

92 Connecting component

94 Thread

96 Connecting piece

98 Contact element

100 Base body

102, 104 Cavity

106 Transfer needle

108 hollow body

110 distal end

112 needle holder

114 hollow needle

116 bearing sleeve

118 needle guard

120 adapter

122 Infusion bag

124 Connector

126 Connecting component

128 Thread

130 Connecting piece

132 Contact element

134 Basic body

136, 138 cavity

Connecting piece

142 media channel

144 Free end

1. A connection component for a medical syringe with a needle system comprising a number of hollow needles arranged in, a tubular connection piece, with an elastic needle cap fastened inside the tubular connection piece and sealingly surrounding the distal tip(s) of each hollow needle of the needle system in an unloaded state, and with a flange jacket which surrounds the tubular connection piece and can rotate about the tubular connection piece and which is provided with a thread for connection to a further bonding component.

2. The connection component of claim 1, the needle system of which, is designed as a double needle system with a first hollow needle provided for a liquid transfer and with a second hollow needle provided for a gas transfer.

3. The connection component of claim 1, the flange jacket of which is rotatable about the needle system and about the elastic needle cap enclosing the needle system.

4. A syringe provided at its distal end with the tubular connection piece of claim 1.

5. A connecting component, in particular for a container for medical substances or medicaments or for a transfer system for medical substances or medicaments, for connection to the connection component of claim 1, with a connecting socket provided with a thread, in which a closing plug consisting of a polypropylene is arranged.

6. A container for medical substances or medicaments with the connecting component of claim 5.

7. The container of claim 6, comprising an outer cap surrounding the connecting component and provided with a disposable closure.

8. A fluid transfer system for transferring medical substances or drugs, comprising the connection component of claim 5 and, adapted thereto with respect to dimensioning and design of the connection pieces, a the connecting, component of claim 5.
